SEQUENCE LISTING (14 sequences)

SEQ ID NO: 1 - DNA, 73 bp, synthetic construct
  source 1..73: mol_type = other DNA; note = Peptide A37.1
ccctgtactggctaggaattcacgttttaatctgggctttgggttaagaaactccccgtgcaataacgatgca

SEQ ID NO: 2 - DNA, 74 bp, synthetic construct
  source 1..74: mol_type = other DNA; note = Peptide A37.2
cgctggaggcgcatcaccgtttgcgtatgtgttctgtgcggcctgccgtcccgtgtgggcaggaacgtcatgga

SEQ ID NO: 3 - DNA, 58 bp, synthetic construct
  source 1..58: mol_type = other DNA; note = Peptide B37.1
cggtgatgcgcctccagcgcggggagtttcttaaccctttccgacttacaagagccgg

SEQ ID NO: 4 - DNA, 74 bp, synthetic construct
  source 1..74: mol_type = other DNA; note = Peptide B37.2
gcgagactcaggtggtgcctttggcattcgaccaggagatatcgcgttcagctatgccccaggaacgtcatgga

SEQ ID NO: 5 - DNA, 59 bp, synthetic construct
  source 1..59: mol_type = other DNA; note = Peptide C37.1
cccatgagaataataccgccgatttacgtcagtccggtttcccacacgggacggcaggc

SEQ ID NO: 6 - DNA, 73 bp, synthetic construct
  source 1..73: mol_type = other DNA; note = Peptide C37.2
cgcacagaacacatacgctttgggcatagctgaacgcgatatctcctggtcgaatgcccaggaacgtcatgga

SEQ ID NO: 7 - DNA, 59 bp, synthetic construct
  source 1..59: mol_type = other DNA; note = Peptide D37.1
gcccagattaaaacgtgaattcctagccagtacagggtttccggactgacgtaaatcgg

SEQ ID NO: 8 - DNA, 73 bp, synthetic construct
  source 1..73: mol_type = other DNA; note = Peptide D37.2
cggtattattctcatgggtttggcaccacctgagtctcgcccggctcttgtaagtcggcaggaacgtcatgga

SEQ ID NO: 9 - DNA, 20 bp, synthetic construct
  source 1..20: mol_type = other DNA; note = SH-DNA (CpG)
tccatgacgttcctgacgtt

SEQ ID NO: 10 - DNA, 21 bp, synthetic construct
  source 1..21: mol_type = other DNA; note = ORF3a-linker-3
tgcatcgttattgcaatatat

SEQ ID NO: 11 - protein, 9 aa, synthetic construct
  source 1..9: note = S3404-412
GDEVRQIAP

SEQ ID NO: 12 - protein, 6 aa, synthetic construct
  source 1..6: note = S4440-445
NLDSKV

SEQ ID NO: 13 - protein, 419 aa, synthetic construct
  source 1..419: note = An amino acid sequence of the N protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 14 - protein, 15 aa, synthetic construct
  source 1..15: note = ORF3a266-280
EPIYDEPTTTTSVPL

(19) United States
(12) Patent Application Publication
(10) Pub. No.: US 2026/0027198 A1
(43) Pub. Date: Jan. 29, 2026

(54) VIRUS POLYPEPTIDE-PROTEIN SUBUNIT COMBINATION VACCINE BASED ON DNA NANOTECHNOLOGY, AND PREPARATION METHOD THEREFOR AND USE THEREOF

(71) Applicants: Xiangfu Laboratory, Jiaxing City (CN); Shanghai University, Shanghai (CN)

(72) Inventors: Lele SUN, Shanghai (CN); Yanfei QU, Shanghai (CN); Yunlong ZHANG, Shanghai (CN); Yaling ZHANG, Shanghai (CN); Lingling YAN, Shanghai (CN); Qinglong YAN, Jiaxing City (CN)

(21) Appl. No.: 19/000,843

(22) Filed: Dec. 24, 2024

(30) Foreign Application Priority Data
Dec. 29, 2023 (CN) 202311865001.8

(51) Int. Cl. A61K 39/215; A61K 38/17; A61K 39/00; A61K 39/145; A61K 39/155; A61K 39/21

(52) U.S. Cl. CPC A61K 39/215; A61K 38/395; A61K 39/145; A61K 39/155; A61K 39/21; A61K 2039/55516; A61K 2039/6025; A61K 2039/645

(57) ABSTRACT

Provided are a virus peptide-protein subunit combination vaccine based on DNA nanotechnology, and a preparation method therefor and use thereof, which belong to the technical field of biological products. Provided is a virus peptide-protein subunit combination vaccine based on DNA nanotechnology, in which a tetrahedral framework nucleic acid formed by assembly of DNA strands is used as a vector, a protein antigen triggering virus-specific T cell activation is coupled to one edge of the tetrahedral framework nucleic acid, and B cell epitope peptides of a virus are coupled to four vertices of the tetrahedral framework nucleic acid.

### CROSS REFERENCE TO RELATED APPLICATION

This patent application claims the benefit and priority of Chinese Patent Application No. 202311865001.8 filed with the China National Intellectual Property Administration on Dec. 29, 2023, the disclosure of which is incorporated by reference herein in its entirety as part of the present application.

### REFERENCE TO SEQUENCE LISTING

A computer readable XML file entitled “GWP20241207855”, that was created on Jan. 21, 2025, with a file size of about 16, 429 bytes, contains the sequence listing for this application, has been filed with this application, and is hereby incorporated by reference in its entirety.

### TECHNICAL FIELD

The present disclosure belongs to the technical field of biological products, and in particular relates to a virus polypeptide-protein subunit combination vaccine based on DNA nanotechnology, a preparation method therefor and use thereof.

### BACKGROUND

Peptide vaccines constructed from antigenic epitopes of pathogens can induce more precise immune responses against pathogenic microorganisms. In addition, peptide vaccines are prepared by chemical means and feature low production costs, and the composition of the vaccines can be quickly adjusted according to the mutation information of pathogens, thereby quickly responding to the damage caused by mutations in pathogens. Since the difference in B cell receptors is at the cellular level, for B cell epitopes, B cell epitopes of one pathogen may cause B cell activation in different individuals. However, due to individual differences in the human major histocompatibility complex, which is responsible for presenting T cell epitopes, it is difficult to screen T cell epitopes (including CD4⁺ and CD8⁺ T cell epitopes) that effectively induce antigen-specific T lymphocyte activation of pathogens in most humans. Therefore, peptide vaccines are currently mainly constructed using B cell epitopes. However, it is difficult to induce efficient humoral immune responses relying solely on B cell epitopes. This is because activation of B cells to form antibody-secreting cells requires not only multivalent antigens to initiate the cross-linking of B cell receptors, but also activated TH2-type helper CD4⁺ T cells to provide B cells with CD40 ligands, but B cell epitopes generally fail to induce T cell activation.

Currently, to improve the immunogenicity of B cell epitopes, B cell epitopes are typically coupled to exogenous carrier proteins (e.g., OVA, BSA, KLH) through cross-linking agents, and CD4⁺ T cells are activated by T-cell epitopes in the carrier proteins, thereby inducing effective humoral immunity. Moreover, it has been shown that combining B cell epitopes of pathogens with a so-called universal T cell epitope can effectively enhance humoral immunity.

Although peptide vaccines constructed in the manner described above are likely to elicit a wide range of humoral immune responses, they cannot induce the activation of pathogen antigen-specific CD4⁺T cells, and thus cannot produce memory CD4⁺T lymphocytes against pathogens, affecting the prophylactic effect of the vaccines. In addition, pathogen-specific killer CD8⁺ T cells can effectively inhibit viral replication by killing virus-infected cells, and are also critical for resisting viral transmission. It has even been shown that Th1-biased immune responses (which assist in CD8⁺ T cell activation) are more favorable for resisting viruses. However, peptide vaccines cannot induce extensive pathogen-specific CD8⁺T cell activation. In response to the issues described above, one possible strategy is to integrate B cell epitopes from pathogens with protein subunits rich in T cell epitopes. However, if the B cell epitopes are directly cross-linked with the pathogenic protein antigens, in one aspect, T cell epitopes of the antigens may be destroyed, and in another aspect, the lack of delivery vectors will affect the efficiency of the presentation of the T cell epitopes of the protein antigens, especially antigen cross-presentation, which is crucial for the activation of CD8⁺ T cells.

### SUMMARY

In view of this, it is an objective of the present disclosure to provide a virus peptide-protein subunit combination vaccine based on DNA nanotechnology. A framework nucleic acid is used as a delivery vector, and B cell epitopes from a virus and a protein subunit are precisely reorganized to construct a universally applicable novel vaccine that can simultaneously induce efficient humoral immunity and cellular immunity.

The present disclosure provides a virus peptide-protein subunit combination vaccine based on DNA nanotechnology, in which a tetrahedral framework nucleic acid formed by assembly of DNA strands is used as a vector, a protein antigen triggering virus-specific T cell activation is coupled to one edge of the tetrahedral framework nucleic acid, and B cell epitope peptides of a virus are coupled to four vertices of the tetrahedral framework nucleic acid.

In some embodiments, nucleotide sequence of the DNA strands for assembly of a tetrahedral framework nucleic acid are set forth in SEQ ID NO: 1 to SEQ ID NO: 8.

In some embodiments, the B cell epitope peptides of a virus are hybridized, via a sulfhydryl-modified DNA strand, with a single-stranded DNA protruding from the vertices of the tetrahedral framework nucleic acid;
the nucleotide sequence of the sulfhydryl-modified DNA strand is set forth in SEQ ID NO: 9.

In some embodiments, the protein antigen triggering virus-specific T cell activation is hybridized, via a DNA strand coupled with it, with a single-stranded DNA protruding from the edge of the tetrahedral framework nucleic acid;
a nucleotide sequence of the coupled DNA strand is set forth in SEQ ID NO: 10.

In some embodiments, the protein antigen triggering virus-specific T cell activation and the DNA strand are coupled via a Halo tag and a Halo ligand.

In some embodiments, the virus includes at least one of the following viruses: a coronavirus, a human immunodeficiency virus peptide vaccine, a respiratory syncytial virus peptide vaccine and an influenza virus.

In some embodiments, the coronavirus includes a novel coronavirus and a variant thereof;
a protein antigen triggering virus-specific T cell activation in the novel coronavirus or the variant thereof includes an N protein;
a B cell epitope peptide of the novel coronavirus or the variant thereof includes S3₄₀₄₋₄₁₂ and/or S4₄₄₀₋₄₄₅.

The present disclosure provides a preparation method for the virus peptide-protein subunit combination vaccine based on DNA nanotechnology, which preparation method includes the following steps:
performing a first hybridization reaction on DNA strands for assembly of a tetrahedral framework nucleic acid to obtain a tetrahedral framework nucleic acid;
mixing a sulfhydryl-modified DNA strand with a B cell epitope peptide of a maleimide-modified virus and performing a coupling reaction to obtain a DNA-coupled B cell epitope peptide;
subjecting a Halo tag-modified protein antigen triggering virus-specific T cell activation to covalent coupling with a Halo ligand-modified DNA strand to obtain a DNA-coupled protein antigen;
subjecting the tetrahedral framework nucleic acid, the DNA-coupled B cell epitope peptide and the DNA-coupled protein antigen to a second hybridization reaction to obtain the virus peptide-protein subunit combination vaccine.

In some embodiments, in the first hybridization reaction, the DNA strands for assembly of a tetrahedral framework nucleic acid are mixed at an equimolar ratio, treated at 95° C. for 10 min, and then maintained at 4° C. for 30 min; a final concentration of each of the DNA strands for assembly of a tetrahedral framework nucleic acid is independently 0.8-1.2 μM;
in the coupling reaction, a molar ratio of the sulfhydryl-modified DNA strand to the maleimide-modified B cell epitope peptide of a virus is 1: (2-10);
in the covalent coupling, a molar ratio of the Halo tag-modified protein antigen triggering virus-specific T cell activation to the Halo ligand-modified DNA strand is (0.9-1.1):(0.9-1.1);
in the second hybridization reaction, a molar ratio of the tetrahedral framework nucleic acid, the DNA-coupled B cell epitope peptide and the DNA-coupled protein antigen is (0.9-1.1):(3.9-4.1):(0.9-1.1).

The present disclosure provides use of the virus peptide-protein subunit combination vaccine based on DNA nanotechnology or a virus peptide-protein subunit combination vaccine obtained by the preparation method in the preparation of an immunized animal model;
in some embodiments, the immunized animal model includes at least one of: rats, mice and rabbits.

The present disclosure provides a virus peptide-protein subunit combination vaccine based on DNA nanotechnology, in which a tetrahedral framework nucleic acid formed by assembly of DNA strands is used as a vector, a protein antigen triggering virus-specific T cell activation is coupled to one edge of the tetrahedral framework nucleic acid, and B cell epitope peptides of a virus are coupled to four vertices of the tetrahedral framework nucleic acid. By systematic immunological evaluation, the results show that coupling of a SARS-COV-2 N protein to a tetrahedral framework nucleic acid can induce CD4⁺T cell activation in a variety of animal models, including C57, BALB/c mice, rats and rabbits, thereby effectively increasing the immune effect of B cell epitopes, and inducing higher antibody titers and increasing the proportions of memory B cells and T cells. In addition, it is surprisingly found that the integration of the N protein on the tetrahedron can induce stronger activation of CD8⁺T cells than the free N protein, probably because the tetrahedron promotes antigen cross-presentation. As can be seen, in the virus peptide-protein subunit combination vaccine provided by the present disclosure, using a tetrahedral framework nucleic acid as a delivery vector can effectively deliver the N protein to T cells, enhance antigen-specific B cell-T cell binding, and allow B cells to acquire more CD40L stimulation, providing a new idea for constructing an efficient protein vector-based viral peptide vaccine.

### BRIEF DESCRIPTION OF THE DRAWINGS

FIGS. 1A-1C show schematic diagrams of the construction principle of a virus peptide-protein subunit combination vaccine based on DNA nanotechnology provided by the present disclosure;

FIGS. 2A-2B show schematic diagrams of the preparation of a tetrahedral framework nucleic acid structure (2A) and the identification results by electrophoresis (2B);

FIG. 3 shows the results of the coupling experiment of sulfhydryl-modified SH-DNA (CpG) to a B cell epitope peptide of a novel coronavirus;

FIG. 4 shows the electrophoresis results of the coupling experiment of a Halo-tag-carrying N protein to DNA-ligand;

FIGS. 5A-5D show the results of the detection of the ability of virus peptide-protein subunit vaccines to stimulate B cells; where 5A shows a flow cytometry analysis plot of in-vitro B cell proliferation; 5B shows statistical data, and 5C shows a flow cytometry analysis plot of in-vitro B cell proliferation; 5D shows statistical data.

FIGS. 6A-6B show the determination results of antibody titers of virus peptide-protein subunit vaccines in vivo, where a shows a schematic diagram of the immunization protocol and b shows the antibody titers induced by different groups of vaccines.

FIGS. 7A-7C show the determination results of helper T cells in vivo after vaccination with novel coronavirus peptide vaccines, where 7A shows the immunization time line; 7B shows the results of flow cytometry analysis of activated helper T cells after vaccination of mice; 7C shows statistical data.

FIGS. 8A-8C show the determination results of killer T cells in vivo after vaccination with novel coronavirus peptide vaccines, where 8A shows the immunization time line; 8B shows the results of flow cytometry analysis of activated killer T cells after vaccination of mice; 8C shows statistical data.

FIGS. 9A-9B show the determination results of the antibody virus neutralization experiment;

FIGS. 10A-10C show the determination results of memory T cells in mice on day 60 after the first immunization, where 10A shows the immunization time line; 10B shows the results of flow cytometry analysis of memory T cells after vaccination of mice; 10C shows statistical data.

FIGS. 11A-11C show the determination results of memory B cells in mice on day 60 after the first immunization, where 11A shows the immunization time line; 11B shows the results of flow cytometry analysis of memory B cells after vaccination of mice; 11C shows statistical data.

FIGS. 12A-12B show the determination results of antibody titers of BALB/c strain mice after vaccination with novel coronavirus peptide vaccines, where 12A shows a schematic diagram of the immunization protocol and 12B shows the antibody titers induced by different groups of vaccines.

FIGS. 13A-13B show the determination results of antibody titers of SD rats after vaccination with novel coronavirus peptide vaccines, where 13A shows a schematic diagram of the immunization protocol and 13B shows the antibody titers induced by different groups of vaccines.

FIGS. 14A-14B show the determination results of antibody titers of rabbits after vaccination with novel coronavirus peptide vaccines, where a shows 14A schematic diagram of the immunization protocol and 14B shows the antibody titers induced by different groups of vaccines.

### DETAILED DESCRIPTION OF THE EMBODIMENTS

The present disclosure provides a virus peptide-protein subunit combination vaccine based on DNA nanotechnology, in which a tetrahedral framework nucleic acid formed by assembly of DNA strands is used as a vector, a protein antigen triggering virus-specific T cell activation is coupled to one edge of the tetrahedral framework nucleic acid, and B cell epitope peptides of a virus are coupled to four vertices of the tetrahedral framework nucleic acid.

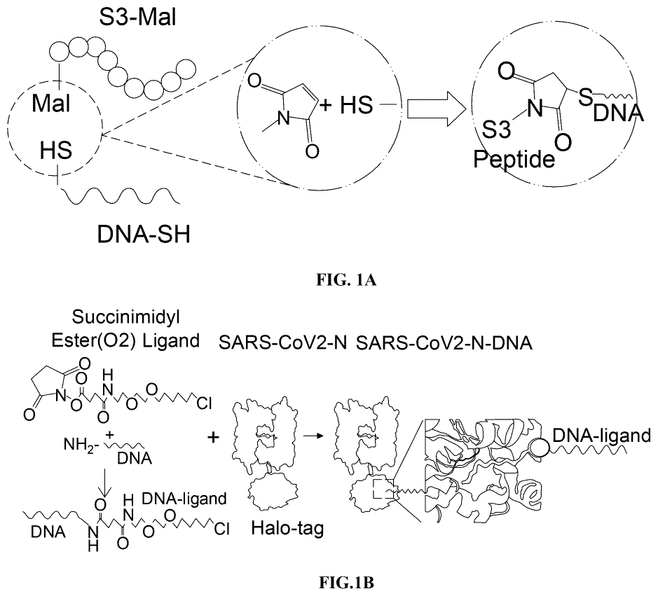

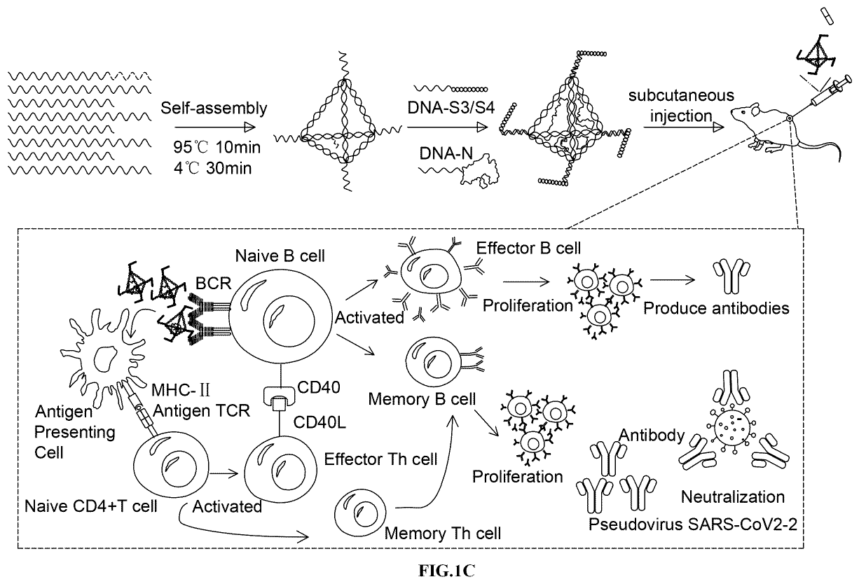

In the present disclosure, by using a self-adjuvant tetrahedral framework nucleic acid, B cell epitopes from a virus and a protein subunit triggering virus-specific T-cell activation are precisely reorganized to construct a universally applicable novel vaccine that can simultaneously induce efficient humoral immunity and cellular immunity. To not damage the T cell epitope, in the present disclosure, a DNA single labeling method is used to modify the protein subunit triggering virus-specific T cell activation, that is, a recombinantly expressed Halo-tag carrying protein subunit triggering virus-specific T cell activation is covalently coupled to a DNA strand modified with a Halo ligand, and then an N protein is assembled by DNA hybridization on one edge of a tetrahedral framework nucleic acid with a side length of 37 base pairs. Meanwhile, B cell epitopes coupled to DNAs by a small molecule cross-linking agent are assembled at vertices of the tetrahedral framework nucleic acid (see FIGS. 1A-1C).

In some embodiments of the present disclosure, nucleotide sequences of the DNA strands for assembly of a tetrahedral framework nucleic acid are as set forth in SEQ ID NO: 1 to SEQ ID NO: 8. In embodiments of the present disclosure, bases 1-37 in A37.1 are paired with bases 1-37 in D37.1 in a complementary manner; bases 40-57 in A37.1 are paired with bases 20-37 in B37.1 in a complementary manner; bases 1-19 in A37.2 are paired with bases 1-19 in B37.1 in a complementary manner; bases 23-40 in A37.2 are paired with bases 1-18 in C37.2 in a complementary manner; bases 41-59 in A37.2 are paired with bases 41-59 in C37.1 in a complementary manner; bases 41-58 in B37.1 are paired with bases 41-58 in D37.2 in a complementary manner; bases 1-19 in B37.2 are paired with bases 22-40 in D37.2 in a complementary manner; bases 23-59 in B37.2 are paired with bases 22-58 in C37.2 in a complementary manner; bases 1-18 in C37.1 are paired with bases 1-18 in D37.2 in a complementary manner; bases 19-37 in C37.1 are paired with bases 41-59 in D37.1 in a complementary manner.

In some embodiments of the present disclosure, the B cell epitope peptides of a virus are hybridized, via a sulfhydryl-modified DNA strand, with a single-stranded DNA protruding from the vertices of the tetrahedral framework nucleic acid; nucleotide sequence of the sulfhydryl-modified DNA strand is set forth in SEQ ID NO: 9. The B cell epitope peptides of the virus achieve covalent cross-linking by chemical reactions between sulfhydryl on the DNA strands and a small molecule cross-linking agent. In some embodiments, the small molecule cross-linking agent is maleimide (see FIG. 1A). The small molecule cross-linking agent is modified on the B cell epitope peptides of the virus. There is no special limitation on the modification method described in the present disclosure, as long as the modification method used is well-known in the art. The single-stranded DNAs protruding from the vertices of the tetrahedral framework nucleic acid are bases 60-74 in A37.2, bases 60-74 in B37.2, bases 59-73 in C37.2, and bases 59-73 in C37.2.

In some embodiments of the present disclosure, the protein antigen triggering virus-specific T cell activation is hybridized, via a DNA strand coupled with it, with a single-stranded DNA on the edge of the tetrahedral framework nucleic acid; a nucleotide sequence of the coupled DNA strand is set forth in SEQ ID NO: 10. In some embodiments, the protein antigen triggering virus-specific T cell activation and the DNA strand are coupled via a Halo tag and a Halo ligand. The single-stranded DNA protruding from the edge of the tetrahedral framework nucleic acid is bases 59-73 in A37.1.

In some embodiments of the present disclosure, the virus includes a coronavirus, a human immunodeficiency virus peptide vaccine, a respiratory syncytial virus peptide vaccine and an influenza virus. In some embodiments, the coronavirus includes a novel coronavirus and a variant thereof; a protein antigen triggering virus-specific T cell activation in the novel coronavirus or the variant thereof includes an N protein. An amino acid sequence of the N protein is set forth in SEQ ID NO: 13 (MSDNGPQNQRNAPRITFGGPSDSTGSNQNGERSGARSKQRRPQGLPNNTASWFTALTQ HGKEDLKFPRGQGVPINTNSSPDDQIGYYRRATRRIRGGDGKMKDLSPRWYFYYLGTGP EAGLPYGANKDGIIWVATEGALNTPKDHIGTRNPANNAAIVLQLPQGTTLPKGFYAEGSR GGSQASSRSSSRSRNSSRNSTPGSSRGTSPARMAGNGGDAALALLLLDRLNQLESKMSG KGQQQQGQTVTKKSAAEASKKPRQKRTATKAYNVTQAFGRRGPEQTQGNFGDQELIRQ GTDYKHWPQIAQFAPSASAFFGMSRIGMEVTPSGTWLTYTGAIKLDDKDPNFKDQVILL NKHIDAYKTFPPTEPKKDKKKKADETQALPQRQKKQQTVTLLPAADLDDFSKQLQQSM SSADSTQA). A B cell epitope peptide of the novel coronavirus or the variant thereof includes S3₄₀₄₋₄₁₂ and/or S4₄₄₀₋₄₄₅. An amino acid sequence of S3₄₀₄₋₄₁₂ is set forth in SEQ ID NO: 11. An amino acid sequence of S4₄₄₀₋₄₄₅ is set forth in SEQ ID NO: 12.

In embodiments of the present disclosure, to verify the immune effect of the T cell epitope peptide and the protein triggering T cell activation in response to different individual differences, a composite vaccine prepared using ORF3a instead of the N protein is also constructed. Specifically, an ORF3a-linker-3 strand is used to form DNA-ORF3a by covalent coupling of sulfhydryl to maleimide, and then a DNA strand is used to hybridize with a single-stranded DNA protruding from an edge of a tetrahedral framework nucleic acid to achieve coupling. The results of the immunological experiments show that due to individual differences in the major histocompatibility complexes of different individuals, ORF3a may not be able to serve as a T cell epitope that effectively induces the activation of antigen-specific CD4 T lymphocytes in BALB/c strain mice, rats and rabbits. For example, TH37-S3-ORF3a & TH37-S4-ORF3a have little effect on further promoting the production of RBD-specific antibodies in mice, rats and rabbits, whereas the coupling of a SARS-COV-2 N protein to a tetrahedral framework nucleic acid can induce higher antibody titers in a variety of animal models, including C57 or BALB/c mice, rats and rabbits.

The present disclosure provides a preparation method for the virus peptide-protein subunit combination vaccine based on DNA nanotechnology, including the following steps:
performing a first hybridization reaction on DNA strands for assembly of a tetrahedral framework nucleic acid to obtain a tetrahedral framework nucleic acid;
mixing a sulfhydryl-modified DNA strand with a B cell epitope peptide of a maleimide-modified virus and performing a coupling reaction to obtain a DNA-coupled B cell epitope peptide;
subjecting a Halo tag-modified protein antigen triggering virus-specific T cell activation to covalent coupling with a Halo ligand-modified DNA strand to obtain a DNA-coupled protein antigen;
subjecting the tetrahedral framework nucleic acid, the DNA-coupled B cell epitope peptide and the DNA-coupled protein antigen to a second hybridization reaction to obtain the virus peptide-protein subunit combination vaccine.

In some embodiments of the present disclosure, in the first hybridization reaction, the DNA strands for assembly of a tetrahedral framework nucleic acid are mixed at an equimolar ratio, treated at 95° C. for 10 min, and then maintained at 4° C. for 30 min. A final concentration of each of the DNA strands for assembly of a tetrahedral framework nucleic acid is independently 0.8-1.2 μM, more preferably 1 μM. In some embodiments, a solvent for the first hybridization reaction is a 1× TAE buffer containing 10 mM magnesium chloride.

In the present disclosure, in the coupling reaction, a molar ratio of the sulfhydryl-modified DNA strand to the maleimide-modified B cell epitope peptide of a virus is 1:(2-10), more preferably 1:3-5. There is no special limitation on the preparation method for the maleimide-modified B cell epitope peptide of a virus in the present disclosure, as long as the preparation method used is well-known in the art. In some embodiments, a temperature of the coupling reaction is 18-27° C., preferably 20-25° C. In some embodiments, a time for the coupling reaction is 10-14 h, preferably 12 h. In some embodiments, the reaction product is purified by ultrafiltration after the coupling reaction is completed.

In the present disclosure, in the covalent coupling, a molar ratio of the Halo tag-modified protein antigen triggering virus-specific T cell activation to the Halo ligand-modified DNA strand is (0.9-1.1):(0.9-1.1), more preferably 1:1. In some embodiments, during the covalent coupling, a reaction temperature is 36-38° C., preferably 37° C. In some embodiments, a reaction time is 10-14 h, preferably 12 h. In the present disclosure,

a method of modifying a protein antigen triggering virus-specific T cell activation with a Halo tag is used, and a plasmid is constructed for expressing a fusion protein. In some embodiments, the Halo ligand-modified DNA strand is prepared by Halo Tag Succinimidyl Ester (O₂) Ligand kit. In some embodiments, a reaction solvent for the covalent coupling is 1× PBS.

In some embodiments of the present disclosure, in the second hybridization reaction, a molar ratio of the tetrahedral framework nucleic acid, the DNA-coupled B cell epitope peptide and the DNA-coupled protein antigen is (0.9-1.1):(3.9-4.1):(0.9-1.1), preferably 1:4:1. In some embodiments, a temperature of the second hybridization reaction is 18-27° C., preferably 20-25° C. In some embodiments, a time for the second hybridization reaction is 20-40 min, preferably 30 min.

The present disclosure provides use of the virus peptide-protein subunit combination vaccine based on DNA nanotechnology or a virus peptide-protein subunit combination vaccine obtained by the preparation method in the preparation of an immunized animal model;
in some embodiments of the present disclosure, the immunized animal model includes at least one of: rats, mice and rabbits.

In the present disclosure, the tetrahedral framework nucleic acids coupled to B cell epitopes can effectively induce B cell proliferation, whereas free peptides and free CpG exhibit very limited ability to stimulate B cell proliferation. It is noteworthy that the vaccine targeting the group of the T cell epitope (ORF3a) of C57 mice and the universal N protein has a stronger stimulating ability. This is due to CD4⁺ T cell activation, which effectively increases the immune effect of B cell epitopes. After animals are immunized with the virus peptide-protein subunit combination vaccine (TH37-S3/S4-N pro) prepared by the present disclosure, they can produce RBD-specific antibodies. Compared to TH37+free S3/S4, TH37-S3/S4, TH37-S3/S4+free N protein, TH37-S3/S4-ORF3a, there is a significant difference in the amount of antibody secreted, and the antibody titers are as high as above 10⁴. This may be attributed to the activation of helper CD4 T cells by the N protein, which effectively promotes the activation and differentiation of B cells. Compared to the free N protein vaccine group, the framework nucleotide can efficiently deliver the N protein to T cells, enhance antigen-specific B cell-T cell binding, and allow B cells to acquire more CD40-L stimulation. Compared to TH37+free S3/S4, TH37-S3/S4, TH37-S3/S4+free N protein, and TH37-S3/S4-ORF3a, CD4⁺ T cells in the peripheral blood of mice are activated at the highest proportion after immunization with TH37-S3/S4-N pro. Moreover, the results of the antibody neutralization experiment show that the antibody in the serum of mice introduced with the N protein vaccine group exhibits the most effective virus neutralization ability. The novel coronavirus subunit-peptide vaccine constructed in the present disclosure can efficiently activate B cells, thereby inducing B cell-mediated humoral immune responses, promoting the production of specific neutralizing antibodies and protecting mice from viral infections. In addition, the novel coronavirus subunit-peptide vaccine constructed by the present disclosure can induce the production of high levels of memory B cells and memory T cells. In addition, it is surprisingly found in the present disclosure that the integration of the N protein on the tetrahedron induces stronger activation of CD8⁺ T cells than the free N protein, possibly because the tetrahedron promotes antigen cross-presentation.

The virus peptide-protein subunit combination vaccine based on DNA nanotechnology, and the preparation method therefor and use thereof provided by the present disclosure will be described in detail below in conjunction with the examples, which, however, should not be construed as limiting the protection scope of the present disclosure.

### EXAMPLE 1

Method for Preparing Virus peptide-Protein Subunit Combination Vaccines Based on DNA Nanotechnology

Experimental reagents: DNA strands used for constructing a framework nucleic acid with protruding strands of an assemblable vaccine are shown in Table 1; other DNA strands used for constructing a virus peptide-protein subunit vaccine are shown in Table 2. All DNA primers were synthesized and purified by Sangon Biotech (Shanghai) Co., Ltd.

B cell epitope peptides in an RBD region of a spike glycoprotein of a novel coronavirus: S3, S4; CD4 T cell epitope peptide: ORF3a. Amino acid sequences of all peptide segments are shown in Table 3. Specifically, they were all synthesized and purified by GenScript Biotech Corporation.

TABLE 1

| Name | Sequence (5′-3′) |
|---|---|
| A37.1 | CCCTGTACTGGCTAGGAATTCACGTTTTA |
| | ATCTGGGCTTTGGGTTAAGAAACTCCCCG |
| | TGCAATAACGATGCA |
| | (SEQ ID NO: 1) |
| A37.2 | CGCTGGAGGCGCATCACCGTTTGCGTATG |
| | TGTTCTGTGCGGCCTGCCGTCCCGTGTGG |
| | GCAGGAACGTCATGGA |
| | (SEQ ID NO: 2) |
| B37.1 | CGGTGATGCGCCTCCAGCGCGGGGAGTTT |
| | CTTAACCCTTTCCGACTTACAAGAGCCGG |
| | (SEQ ID NO: 3) |
| B37.2 | GCGAGACTCAGGTGGTGCCTTTGGCATTC |
| | GACCAGGAGATATCGCGTTCAGCTATGCC |
| | CCAGGAACGTCATGGA |
| | (SEQ ID NO: 4) |
| C37.1 | CCCATGAGAATAATACCGCCGATTTACGT |
| | CAGTCCGGTTTCCCACACGGGACGGCAGG |
| | C |
| | (SEQ ID NO: 5) |
| C37.2 | CGCACAGAACACATACGCTTTGGGCATAG |
| | CTGAACGCGATATCTCCTGGTCGAATGCC |
| | CAGGAACGTCATGGA |
| | (SEQ ID NO: 6) |
| D37.1 | GCCCAGATTAAAACGTGAATTCCTAGCCA |
| | GTACAGGGTTTCCGGACTGACGTAAATCG |
| | G |
| | (SEQ ID NO: 7) |
| D37.2 | CGGTATTATTCTCATGGGTTTGGCACCAC |
| | CTGAGTCTCGCCCGGCTCTTGTAAGTCGG |
| | CAGGAACGTCATGGA |
| | (SEQ ID NO: 8) |

TABLE 2

| Name | Sequence (5′-3′) |
|---|---|
| DNA | TGCATCGTTATTGCAATATAT |
| linker- | (SEQ ID NO: 10) |
| 3 | |
| SH-DNA | T*C*C*A*T*G*A*C*G*T*T* |
| (CpG) | C*C*T*G*A*C*G*T*T |
| | (SEQ ID NO: 9) |
| ORF3a- | TGCATCGTTATTGCAATATAT |
| linker- | (SEQ ID NO: 10) |
| 3 | |

* denotes a phosphorothioate diester bond modification, which is intended to enhance the stability of the DNA strands.

TABLE 3

| Name | Sequence (5′-3′) |
|---|---|
| S3404-412 | GDEVRQIAP |
| | (SEQ ID NO: 11) |
| S4440-445 | NLDSKV |
| | (SEQ ID NO: 12) |
| ORF3a266-280 | EPIYDEPTTTTSVPL |
| | (SEQ ID NO: 14) |

### 1. Preparation of a Framework Nucleic Acid Structure

DNA strands designed in Table 1 were mixed at an equimolar ratio (final concentration of 1 μM) in a 1× TAE (containing 10 mM magnesium chloride) buffer, and the mixture was heated to 95° C. and maintained at this temperature for 10 minutes, and then cooled to 4° C. for annealing. The reaction products were identified by polyacrylamide gel electrophoresis.

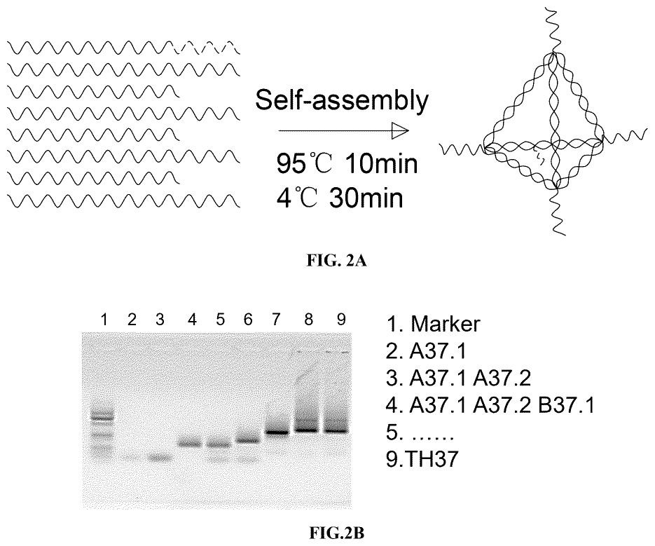

The results are shown in FIG. 2B. It could be seen from the electrophoretogram that a tetrahedral framework nucleic acid structure (TH37) was successfully synthesized.

### 2. Construction of Framework Nucleic Acid-Based Virus Peptide-Protein Subunit Combination Vaccines

To construct a novel coronavirus peptide vaccine based on the tetrahedral framework nucleic acid TH37, sulfhydryl-modified DNA-SH was first coupled to a virus peptide.

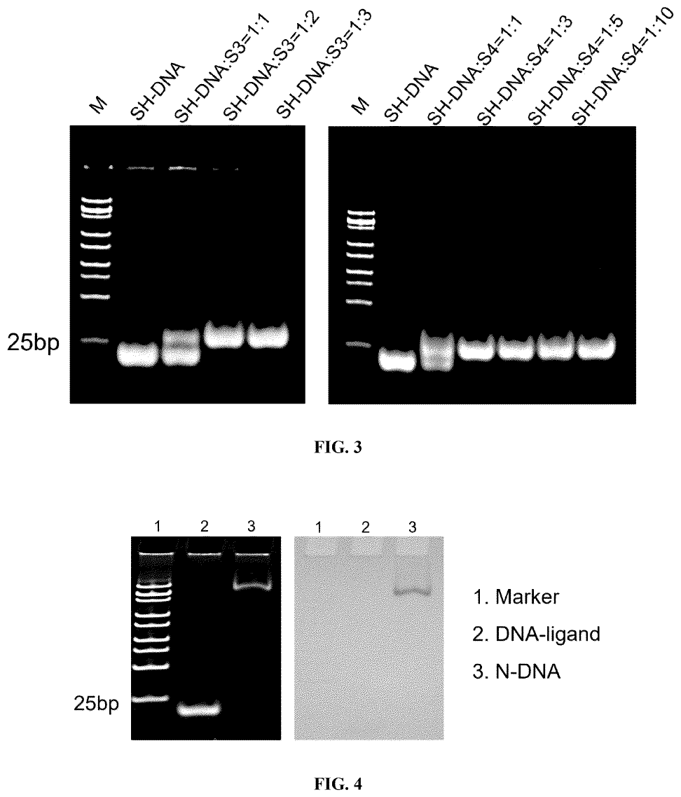

1) For the coupling of the sulfhydryl-modified SH-DNA (CpG) to a B cell epitope peptide S3 or S4 of a novel coronavirus, SH-DNA (CpG) (100 μM) was separately mixed at a molar ratio of 1:3 with maleimide Mal-modified S3 and S4 (5 mM) in PBS, and a resulting mixture was reacted under shaking overnight at room temperature and purified by ultrafiltration to obtain DNA-S3 and DNA-S4. Other experiments with different molar ratios were also separately set up according to the same method, including mixing SH-DNA and S3 at a molar ratio of 1:1 or 1:2; mixing SH-DNA and S4 at molar ratios of 1:1, 1:5 and 1:10. The products after purification by ultrafiltration was subjected to electrophoresis. The electrophoresis results are shown in FIG. 3. It could be seen from FIG. 3 that when SH-DNA and S3 or S4 were reacted at a molar ratio of 1:1, no single band could be formed, indicating that the coupling effect of the two was poor, and when SH-DNA and S3 or S4 were mixed in molar ratios of 1:2 and 1:3, or 1:5 and 1:10, good coupling could be achieved. For economic reasons, in some embodiments, SH-DNA and S3 or S4 were mixed at a molar ratio of 1:2 to 1:5, preferably 1:3.

2) For the coupling of sulfhydryl-modified ORF3a-linker-3 to a CD4 T cell epitope peptide ORF3a of a novel coronavirus, ORF3a-linker-3 (100 μM) was mixed at a molar ratio of 1:10 with maleimide Mal-modified ORF3a (5 mM) in PBS, and a resulting mixture was reacted under shaking overnight at room temperature and purified by ultrafiltration to obtain DNA-ORF3a.

3) For the covalent coupling of a recombinantly expressed Halo-tag-carrying N protein to DNA modified with a Halo ligand:

### Construction of Plasmids:

A DNA sequence (YP_009724397.2 (335Gly/Ala)) (Met1-Ala419) encoding a nucleocapsid protein of severe acute respiratory syndrome coronavirus-2 (2019-nCOV) was synthesized by Transheep (Shanghai, China). A N-terminal TPA signal peptide for secretion was first incorporated into a pcDNA3.1 (+) vector. An N sequence, a Halo-tag sequence (GenBank ADN27525.1) and 8xHis-tag were then subcloned downstream of the TPA signal peptide according to standard molecular techniques.

### Cell Culture and Transfection:

Expi293F cells (Thermo Fisher) were grown in an OPM-293 CD05 medium (OPM Biosciences) at 120 rpm, 8% CO₂, and 37° C. To produce N Halo, the cells were adjusted to 2×10⁶/ml. 1 μg of plasmid and 3 μg of polyethyleneimine (PEI, Polysciences) were mixed in 100 μl of OPM-293 CD05 medium (OPM Biosciences) per ml of culture. 20 hours after the transfection, transfection ProFeed (OPM Biosciences) was added, and the cells were cultured for 3-6 days. The cells were removed by centrifugation at 1000×g for 3 minutes and the following protein purification was performed using a medium.

### Purification of N Halo Protein:

The culture supernatant was injected through a Ni-NTA column (Smart Lifesciences) at 4° C. His8-tagged proteins were eluted by stepwise elution with PBS at pH 8 supplemented with 20 mM, 50 mM, and 250 mM imidazoles. Relevant imidazole fractions were pooled and concentrated using a 30 kD MWCO centrifuge (MilliporeSigma). The proteins were separated on a Superdex™ 200 Increase 16/600 GL column (GE Healthcare) using PBS as a running buffer. After being rapidly frozen in liquid nitrogen, the peak fractions were pooled, concentrated and stored at −80° C.

A dimethyl sulfoxide solution of 40 mM Halo Tag Succinimidyl Ester (02) Ligand was shaken with amino-modified DNA-ligand in a buffer system of 1× PBS at room temperature for 5 hours. After the coupling was successful as characterized by mass spectrometry, the prepared Halo ligand-coupled DNA was mixed with the Halo-tag-carrying N protein in a buffer system of 1×PBS at a molar ratio of 1:1, and the mixture was incubated at 37° C. overnight to obtain DNA-N pro.

The prepared DNA-N pro (N-DNA) and Halo ligand-coupled DNA (DNA-ligand) were subjected to electrophoresis detection. The results are shown in FIG. 4. It could be seen from FIG. 4 that the band formed by Halo ligand-coupled DNA was far from the sample loading well, whereas the band of N-DNA was close to the sample loading well, indicating that the DNA-ligand was successfully coupled to the N protein.

### 4) Assembly of CD4 T Cell Epitope Peptide ORF3a on TH37

The tetrahedral framework nucleic acid TH37 was mixed with DNA-S3 at a molar ratio of 1:4, and hybridized and paired in a complementary manner at room temperature to obtain TH37-S3.

The tetrahedral framework nucleic acid TH37 was mixed with DNA-S4 at a molar ratio of 1:4, and hybridized and paired in a complementary manner at room temperature to obtain TH37-S4.

The tetrahedral framework nucleic acid TH37 was mixed with DNA-S3 and DNA-ORF3a at a molar ratio of 1:4: 1, and hybridized and paired in a complementary manner at room temperature to obtain TH37-S3-ORF3a.

The tetrahedral framework nucleic acid TH37 was mixed with DNA-S4 and DNA-ORF3a at a molar ratio of 1:4:1, and hybridized and paired in a complementary manner at room temperature to obtain TH37-S4-ORF3a.

### 5) Assembly of N Protein on TH37

The tetrahedral framework nucleic acid TH37 was mixed with DNA-S3 and DNA-N pro at a molar ratio of 1:4:1, and hybridized and paired in a complementary manner at room temperature to obtain TH37-S3-N pro.

The tetrahedral framework nucleic acid TH37 was mixed with DNA-S4 and DNA-N pro at a molar ratio of 1:4: 1 to obtain TH37-S4-N pro.

### EXAMPLE 2

### Ability of Framework Nucleic Acid-Based Virus Peptide-Protein Subunit Combination Vaccines to Stimulate B Cells

One C57 mouse was sacrificed. The spleen was taken out and placed in a cell culture dish, and then ground after adding PBS. The crushed spleen suspension was centrifuged at 1500 rpm for 3 minutes to collect the precipitate, and then 2 mL of red blood cell lysis buffer was added. After 3 minutes of lysis, an equal volume of PBS was added to stop the lysis. After centrifugation (1500 rpm, 3 min), splenocytes were obtained by filtration through gauze, resuspended in 2 mL of PBS and counted using a cytometer. Staining was performed using carboxyfluorescein diacetate succinimide ester (CFSE). 1×10⁷ splenocytes were suspended in 5 mL of PBS, and 0.25 μL of 10 mM CFSE in dimethyl sulfoxide was added. The cell suspension was then placed in a cell incubator at 37° C. and stained for 15 minutes. After that, the CFSE-stained splenocytes were collected by centrifugation (1500 rpm, 5 min) and resuspended in a 1640 medium. For the assay of the activation ability against B cells, 5×10⁵ CFSE-stained splenocytes were suspended in 200 μL of 1640 medium, and incubated with a framework nucleic acid-based virus peptide-protein subunit vaccine sample in a round bottom 96-well plate (final concentration: 60 nM framework nucleic acid). After 3 days of culture, the splenocytes were collected by centrifugation (1500 rpm, 3 min). B cell (CD19⁺) proliferation was detected by flow cytometry, and cell proliferation was analyzed by the CFSE fluorescence dilution method.

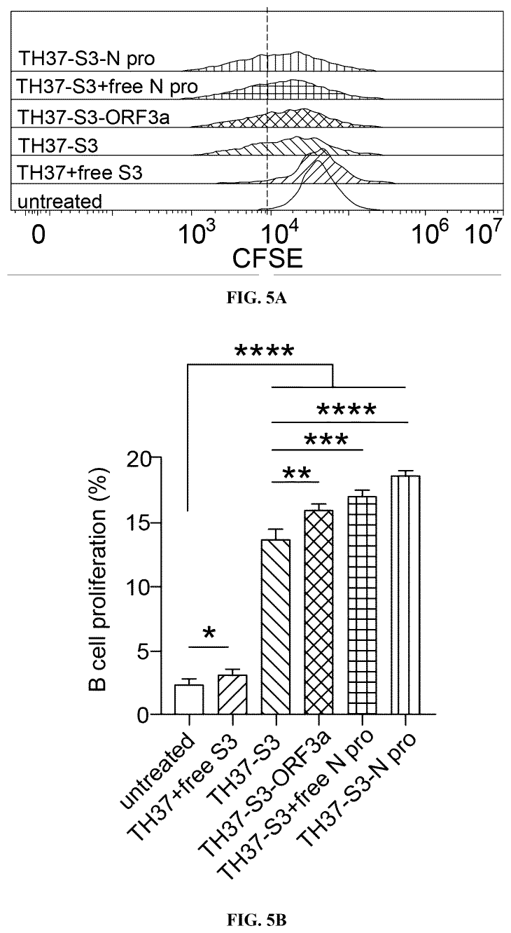

The results are shown in FIGS. 5A-5D. The results showed that the vaccine groups with B cell epitopes all could effectively induce B cell proliferation, whereas free peptide and free CpG exhibited very limited ability to stimulate B cell proliferation. It is noteworthy that the vaccine targeting the group of the T cell epitope of C57 mice and the universal N protein has a stronger stimulating ability. This is due to CD4⁺ T cell activation, which effectively increases the immune effect of B cell epitopes.

### EXAMPLE 3

Determination of Antibody Titers In Vivo After Vaccination with Framework Nucleic Acid-Based Virus Peptide-Protein Subunit Vaccines

For the immunization of mice with the virus peptide-protein subunit vaccines constructed based on TH37, 30 female C57BL/6 mice were randomly divided into 6 groups, and on day 0 and day 14, the mice were immunized subcutaneously on the dorsal side with the following vaccination schemes, respectively:
Scheme I: no vaccination;
Scheme II: TH37 (500 pmol)+free S3 and TH37 (500 pmol)+free S4, designated as TH37+free S3/S4 group;
Scheme III: TH37-S3 (500 pmol)+TH37-S4 (500 pmol), designated as TH37-S3/S4group;
Scheme IV: TH37-S3-ORF3a (500 pmol)+TH37-S4-ORF3a (500 pmol), designated as TH37-S3/S4-ORF3a group;
Scheme V: TH37-S3+free N pro (500 pmol) and TH37-S4+free N pro (500 pmol), designated as TH37-S3/S4 +free N pro group;
Scheme VI: TH37-S3-N pro (500 pmol) and TH37-S4-N pro (500 pmol), designated as TH37-S3/S4-N pro group.

For the determination of specific antibody titers in the serum of mice after vaccination, the RBD protein of a peptide or novel coronavirus S protein was coated in a high-adsorption 96-well Costar plate at 4° C. overnight. After blocking with a blocking buffer, serum samples with different dilution multiples on day 21 were added to the plate for incubation. Subsequently, detection was performed using horseradish peroxidase (HRP)-conjugated goat anti-mouse IgG. Finally, TMB substrate was added for color development, and the absorbances at 450 nm and 620 nm were measured using a microplate reader respectively. The antibody titer of each vaccinated group was measured in comparison with the non-vaccinated group, that is, a signal was considered detectable if the absorbance value was greater than twice the average absorbance of the non-vaccinated group.

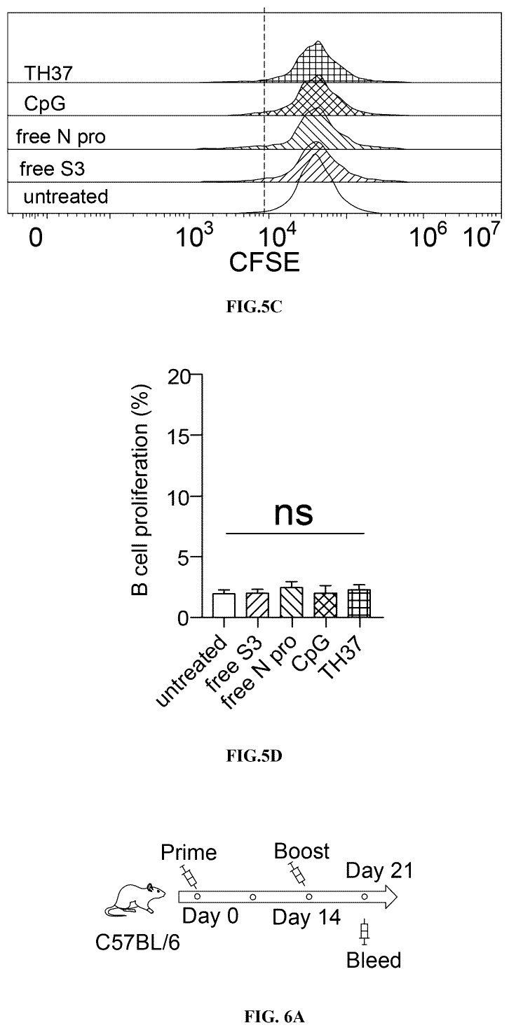

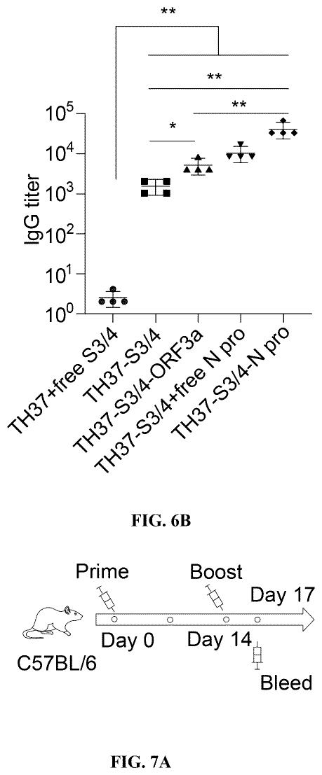

The results are shown in FIGS. 6A-6B. Compared to the mice in the control group, RBD-specific antibodies were detected in mice immunized with a framework nucleic acid-based viral subunit-peptide vaccine. However, only extremely limited specific antibodies could be produced in the mice immunized with free peptides. It is noteworthy that among all the framework nucleic acid-based viral subunit-peptide vaccines, the TH37-S3-N pro and TH37-S4-N pro group exhibits the strongest induction of specific antibodies in vivo, with the antibody titer of more than 10⁴. The above results can be attributed to the activation of helper CD4 T cells by the N protein, which effectively promotes the activation and differentiation of B cells. Compared to the free N protein vaccine group, the framework nucleic acid can efficiently deliver the N protein to T cells, enhance antigen-specific B cell-T cell binding, and allow B cells to acquire more CD40L stimulation.

### EXAMPLE 4

Activation of Helper T Cells In vivo After Vaccination with Novel Coronavirus Peptide Vaccines

Mice were vaccinated with a framework nucleic acid-based novel coronavirus subunit-peptide vaccine (TH37-S3/S4-N pro, TH37-S3/S4+free N pro), a framework nucleic acid-based novel coronavirus peptide vaccine (TH37-S3/S4-ORF3a, TH37-S3/S4), and TH37+free S3/S4, respectively, using the following vaccination schemes. The specific schemes were as follows:
Scheme I: no vaccination;
Scheme II: TH37 (500 pmol)+free S3 and TH37 (500 pmol)+free S4, designated as TH37+free S3/S4 group;
Scheme III: TH37-S3 (500 pmol)+TH37-S4 (500 pmol), designated as TH37-S3/S4group;
Scheme IV: TH37-S3-ORF3a (500 pmol)+TH37-S4-ORF3a (500 pmol), designated as TH37-S3/S4-ORF3a group;
Scheme V: TH37-S3+free N pro (500 pmol) and TH37-S4+free N pro (500 pmol), designated as TH37-S3/S4+free N pro group;
Scheme VI: TH37-S3-N pro (500 pmol) and TH37-S4-N pro (500 pmol), designated as TH37-S3/S4-N pro group.

The activation level of CD4⁺ T cells in mice was determined. The mice were immunized on day 0 and day 14, and peripheral blood was taken on day 21. Single cell suspensions obtained after the lysis of red blood cells were detected for the proportion of activated CD4 T cells (IL-4 in CD3⁺CD4⁺) by flow cytometry.

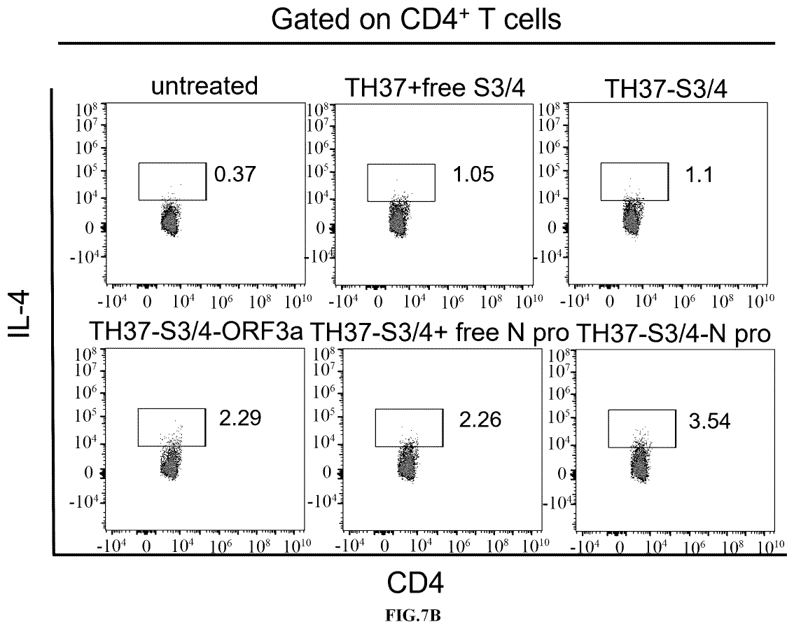

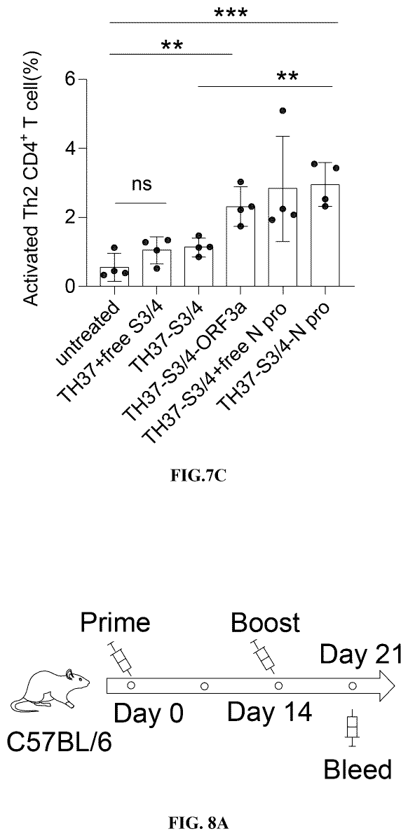

The results are shown in FIGS. 7A-7C. It was seen from the flow cytometry results and the corresponding statistical results that after treatment with TH37-S3/S4-N pro, CD4 T cells in the peripheral blood of mice were activated at the highest proportion, which was consistent with the results of B cell proliferation described above.

### EXAMPLE 5

Activation of Killer T Cells In vivo After Vaccination with Novel Coronavirus Peptide Vaccines

For determination of the activation level of CD8⁺ T cells in mice after vaccination with framework nucleic acid-based novel coronavirus peptide vaccines, the mice were immunized on day 0 and day 14, and peripheral blood was taken on day 21. Single cell suspensions obtained after the lysis of red blood cells were detected for the proportion of activated CD8⁺ T cells (IFN-γ in CD3⁺CD8⁺) by flow cytometry.

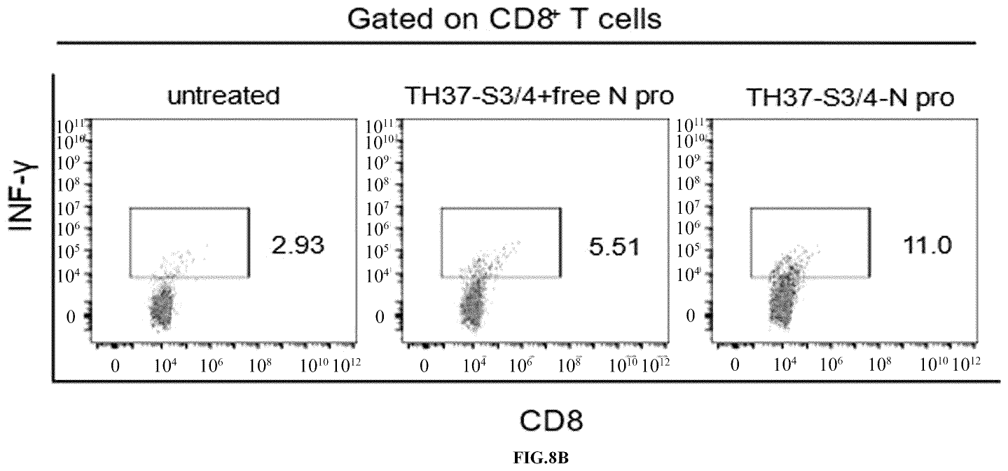

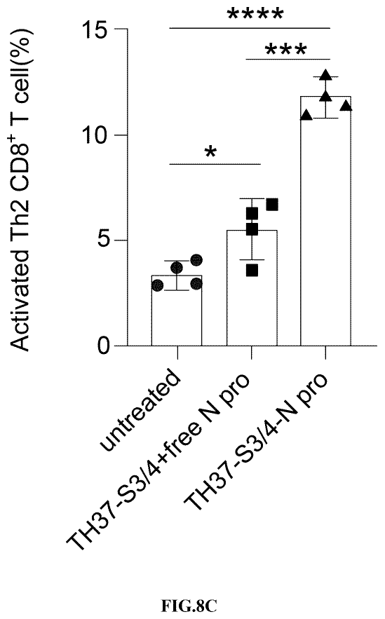

The results are shown in FIGS. 8A-8C. It was surprisingly found that the integration of the N protein on the tetrahedron could induce stronger activation of CD8⁺ T cells than the free N protein, probably because the tetrahedron promoted antigen cross-presentation.

### EXAMPLE 6

### Antibody Virus Neutralization Experiment

HEK-293T (HEK-293T-ACE2) cells used in the antibody virus neutralization experiment, which were transfected with a receptor ACE2 binding to the RBD region of the novel coronavirus S protein, were cultured in a DMEM high-glucose medium containing 10% FBS and 1% diabody under the standard conditions of 37° C. and 5% CO₂. The experiment used GFP expression in pseudovirus-SARS-COV-2, and observed and analyzed it by flow cytometry. The specific scheme was as follows. 5×10⁴ HEK-293T-ACE2 cells were inoculated in a 24-well plate, then a mixed sample obtained by incubating 1 μL of the novel coronavirus pseudovirus-SARS-COV-2 with the serum sample on day 21 of immunization in Example 3 at 37° C. for 1 h was added to the well plate and incubated with the cells. After 12 h, the samples were washed away, and a fresh DMEM high-glucose medium was added for further culture for 24 h. Finally, centrifugation was performed to discard the supernatant, and the cells were resuspended in a DMEM high-glucose medium. The expression of green fluorescence of GFP was observed under a flow cytometer.

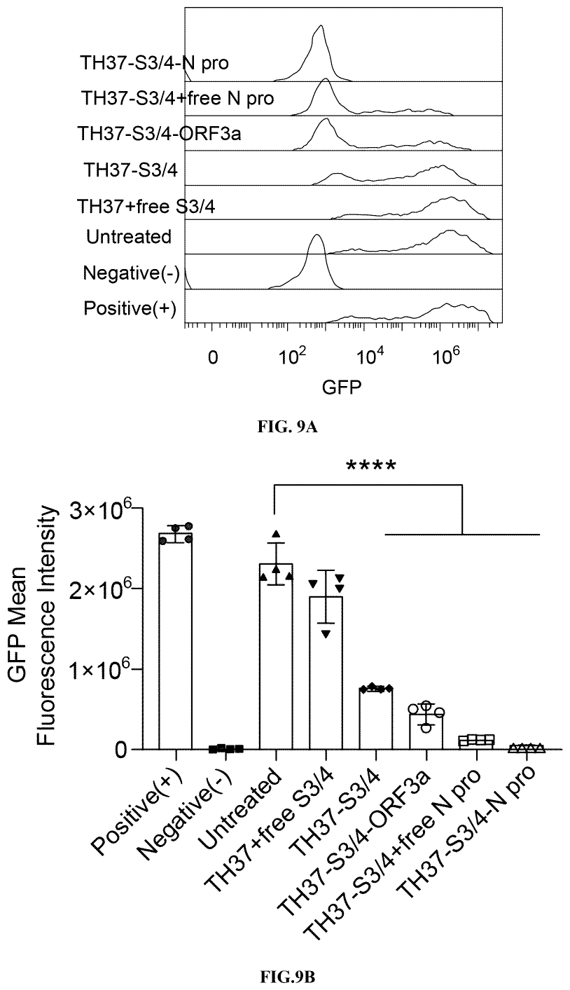

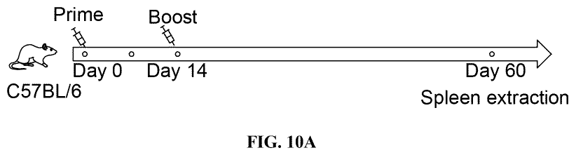

The results are shown in FIGS. 9A-9B. It is noteworthy that the antibody in the serum of mice introduced with the N protein vaccine group exhibits the most effective virus neutralization ability. The above results together demonstrate that the TH37-based novel coronavirus subunit-peptide vaccine constructed can effectively activate B cells, thereby inducing B cell-mediated humoral immune responses, promoting the production of specific neutralizing antibodies and protecting mice from viral infections.

### EXAMPLE 7

### Evaluation of Immune Memory Effect

The immune memory effect in mice was detected on day 60 after the first immunization. Mice were sacrificed and spleens were treated by the method described above to obtain single cell suspensions, which were stained with flow cytometry antibodies and then detected for the levels of memory T cells (CD3⁺CD4⁺CD44highCD62Lhigh) and memory B cells (CD19⁺CD27⁺) by a flow cytometer.

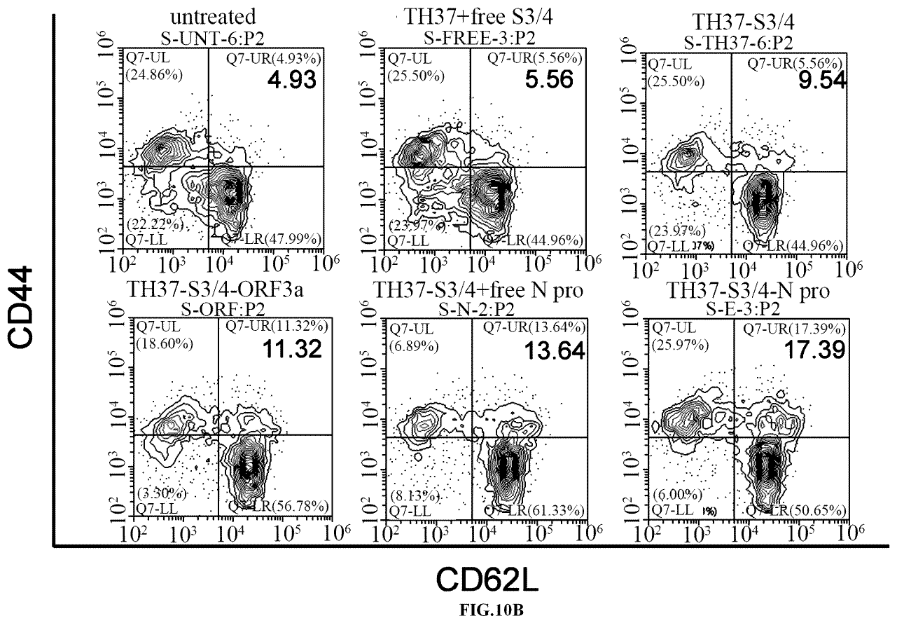

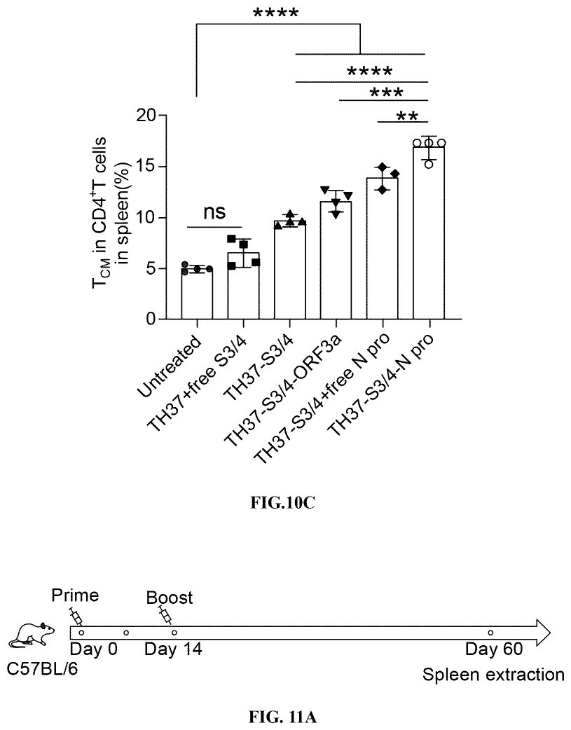

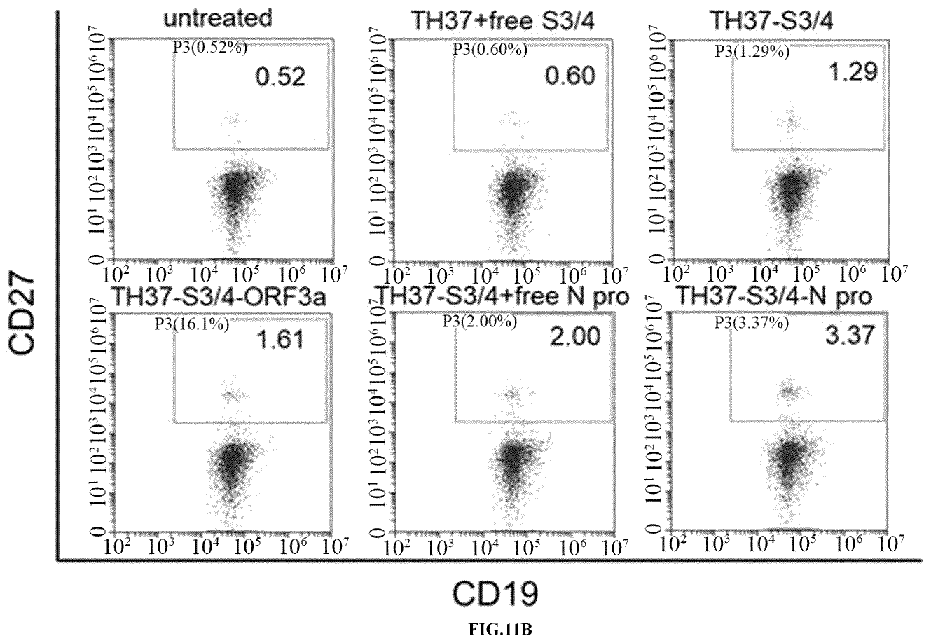

The results are shown in FIGS. 10A-11C. Memory T cells and memory B cells were produced in all vaccinated mice, and the proportion of memory cells was further increased in the mice immunized with the group containing the CD4 T epitope peptide and the universal N protein.

### EXAMPLE 8

Advantage Comparison of Virus peptide-Protein Subunit Vaccines Constructed Based on Framework Nucleic Acid

To demonstrate the advantages of the constructed framework nucleic acid-based virus peptide-protein subunit vaccines, BALB/c strain mice were immunized. Experimental animals were randomly divided into 5 groups, and on day 0 and day 14, the animals were immunized subcutaneously on the dorsal side with (I) no inoculation, (II) TH37 +free S3 (500 pmol TH37) and TH37+free S4 (500 pmol), (III) TH37-S3 (500 pmol) and TH37-S4 (500 pmol), (IV) TH37-S3-ORF3a (500 pmol) and TH37-S4-ORF3a (500 pmol), and (V) TH37-S3-N pro (500 pmol) and TH37-S4-N pro (500 pmol), respectively.

Rats and rabbits were immunized with TH37-S3-ORF3a & TH37-S4-ORF3a and TH37-S3-N pro & TH37-S4-N pro, respectively. Experimental animals were randomly divided into 3 groups, and on day 0 and day 14, the animals were immunized subcutaneously on the dorsal side with (I) no inoculation, (II) TH37-S3-ORF3a & TH37-S4-ORF3a, and (III) TH37-S3-N pro & TH37-S4-N pro, respectively.

The RBD-specific antibody titers in the serum after vaccination were determined by the method described in Example 3. ORF3a is a CD4⁺ T cell epitope specific for C57 mice. However, due to individual differences in the major histocompatibility complexes of different individuals, ORF3a may not be able to serve as a T cell epitope that effectively induces the activation of antigen-specific T lymphocytes in BALB/c strain mice, rats and rabbits.

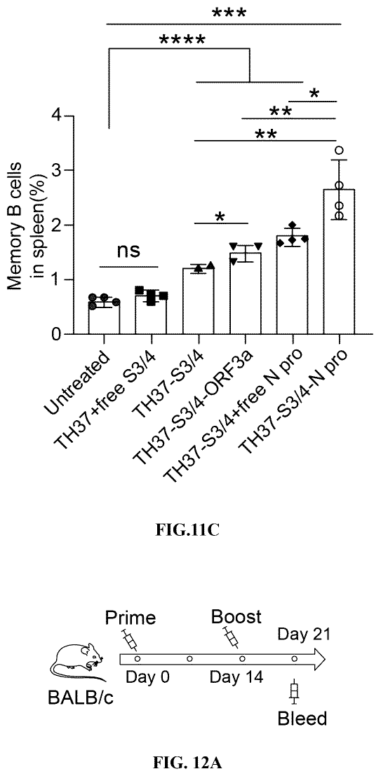

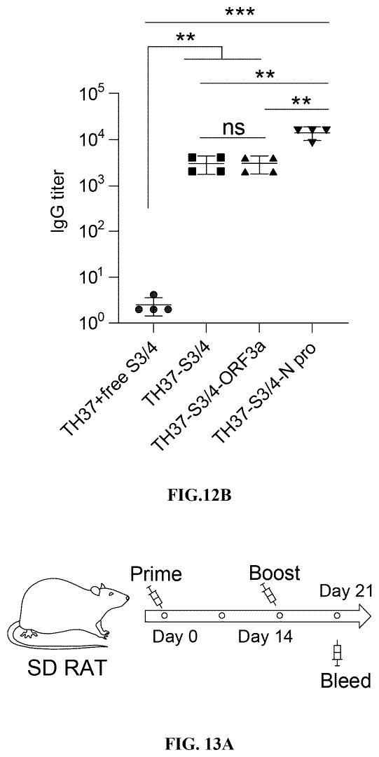

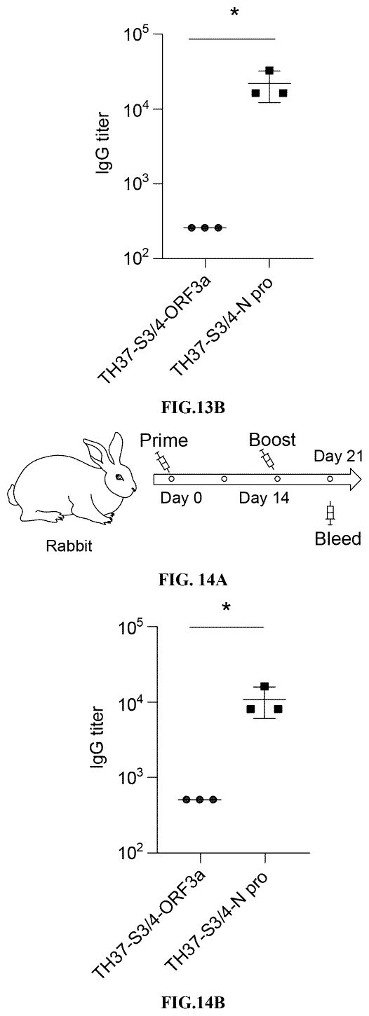

The results are shown in FIGS. 12A-14B. The results showed that TH37-S3-ORF3a & TH37-S4-ORF3a had little effect on further promoting the production of RBD-specific antibodies in mice, rats and rabbits, whereas the coupling of a SARS-COV-2 N protein to a tetrahedral framework nucleic acid could induce higher antibody titers in a variety of animal models, including C57, BALB/c, rat and rabbits.

The description above is only the preferred embodiments of the present disclosure. It should be noted that several improvements and modifications may also be made by those of ordinary skill in the art without departing from the principle of the present disclosure, and these improvements and modifications shall also be considered within the protection scope of the present disclosure.

1. A virus peptide-protein subunit combination vaccine based on DNA nanotechnology, wherein a tetrahedral framework nucleic acid formed by assembly of DNA strands is used as a vector, a protein antigen triggering virus-specific T cell activation is coupled to one edge of the tetrahedral framework nucleic acid, and B cell epitope peptides of a virus are coupled to four vertices of the tetrahedral framework nucleic acid.

2. The virus peptide-protein subunit combination vaccine based on DNA nanotechnology according to claim 1, wherein nucleotide sequences of the DNA strands for assembly of a tetrahedral framework nucleic acid are set forth in SEQ ID NO: 1 to SEQ ID NO: 8.

3. The virus peptide-protein subunit combination vaccine based on DNA nanotechnology according to claim 1, wherein the B cell epitope peptides of a virus are hybridized, via a sulfhydryl-modified DNA strand, with a single-stranded DNA protruding from the vertices of the tetrahedral framework nucleic acid;
the nucleotide sequence of the sulfhydryl-modified DNA strand is set forth in SEQ ID NO: 9.

4. The virus peptide-protein subunit combination vaccine based on DNA nanotechnology according to claim 1, wherein the protein antigen triggering virus-specific T cell activation is hybridized, via a DNA strand coupled with it, with a single-stranded DNA protruding from the edge of the tetrahedral framework nucleic acid;
the nucleotide sequence of the coupled DNA strand is set forth in SEQ ID NO: 10.

5. The virus peptide-protein subunit combination vaccine based on DNA nanotechnology according to claim 4, wherein the protein antigen triggering virus-specific T cell activation and the DNA strand are coupled via a Halo tag and a Halo ligand.

6. The virus peptide-protein subunit combination vaccine based on DNA nanotechnology according to claim 1, wherein the virus comprises at least one of the following viruses: a coronavirus, a human immunodeficiency virus peptide vaccine, a respiratory syncytial virus peptide vaccine and an influenza virus.

7. The virus peptide-protein subunit combination vaccine based on DNA nanotechnology according to claim 6, wherein the coronavirus comprises a novel coronavirus and a variant thereof;
a protein antigen triggering virus-specific T cell activation in the novel coronavirus or the variant thereof comprises an N protein;
a B cell epitope peptide of the novel coronavirus or the variant thereof comprises S3₄₀₄₋₄₁₂ and/or S4₄₄₀₋₄₄₅.

8. A method for preparing the virus peptide-protein subunit combination vaccine based on DNA nanotechnology according to claim 1, comprising the following steps:
performing a first hybridization reaction on DNA strands for assembly of a tetrahedral framework nucleic acid to obtain a tetrahedral framework nucleic acid;
mixing a sulfhydryl-modified DNA strand with a B cell epitope peptide of a maleimide-modified virus and performing a coupling reaction to obtain a DNA-coupled B cell epitope peptide;
subjecting a Halo tag-modified protein antigen triggering virus-specific T cell activation to covalent coupling with a Halo ligand-modified DNA strand to obtain a DNA-coupled protein antigen;
subjecting the tetrahedral framework nucleic acid, the DNA-coupled B cell epitope peptide and the DNA-coupled protein antigen to a second hybridization reaction to obtain the virus peptide-protein subunit combination vaccine.

9. The preparation method according to claim 8, wherein in the first hybridization reaction, the DNA strands for assembly of a tetrahedral framework nucleic acid are mixed at an equimolar ratio, treated at 95° C. for 10 min, and then maintained at 4° C. for 30 min; a final concentration of each of the DNA strands for assembly of a tetrahedral framework nucleic acid is independently 0.8-1.2 μM;
in the coupling reaction, a molar ratio of the sulfhydryl-modified DNA strand to the maleimide-modified B cell epitope peptide of a virus is 1:(2-10);
in the covalent coupling, a molar ratio of the Halo tag-modified protein antigen triggering virus-specific T cell activation to the Halo ligand-modified DNA strand is (0.9-1.1):(0.9-1.1);
in the second hybridization reaction, a molar ratio of the tetrahedral framework nucleic acid, the DNA-coupled B cell epitope peptide and the DNA-coupled protein antigen is (0.9-1.1):(3.9-4.1):(0.9-1.1).

10. (canceled)

11. The method according to claim 8, wherein nucleotide sequences of the DNA strands for assembly of a tetrahedral framework nucleic acid are set forth in SEQ ID NO: 1 to SEQ ID NO: 8.

12. The method according to claim 8, wherein the B cell epitope peptides of a virus are hybridized, via a sulfhydryl-modified DNA strand, with a single-stranded DNA protruding from the vertices of the tetrahedral framework nucleic acid;
the nucleotide sequences of the sulfhydryl-modified DNA strands are set forth in SEQ ID NO: 9.

13. The method according to claim 8, wherein the protein antigen triggering virus-specific T cell activation is hybridized, via a DNA strand coupled with it, with a single-stranded DNA protruding from the edge of the tetrahedral framework nucleic acid;
the nucleotide sequence of the coupled DNA strand is set forth in SEQ ID NO: 10.

14. The method according to claim 13, wherein the protein antigen triggering virus-specific T cell activation and the DNA strand are coupled via a Halo tag and a Halo ligand.

15. The method according to claim 8, wherein the virus comprises at least one of the following viruses: a coronavirus, a human immunodeficiency virus peptide vaccine, a respiratory syncytial virus peptide vaccine and an influenza virus.

16. The method according to claim 15, wherein the coronavirus comprises a novel coronavirus and a variant thereof;
a protein antigen triggering virus-specific T cell activation in the novel coronavirus or the variant thereof comprises an N protein;
a B cell epitope peptide of the novel coronavirus or the variant thereof comprises S3₄₀₄₋₄₁₂ and/or S4₄₄₀₋₄₄₅.
